(19) United States
(12) Patent Application Publication
(10) Pub. No.: US 2005/0113309 A1
(43) Pub. Date: May 26, 2005

(54) Crystalline forms(2s)-n-5[amino(imino)methyl]-2-thienylmethyl-1-(2r)-2[(carboxymethyl) amino]-3,3-diphenylpropanoyl-2-pyrrolidinecarboxamide nh2o

(76) Inventors: Aeri Kim, Taejeon (KR); Jae-Hyeon Park, Taejeon (KR); Suk-Kyoon Yoon, Taejeon (KR); Bong-Chan Kim, Taejeon (KR); Sung-Ji Kim, Taejeon (KR); Kwan-Hyung Cho, Taejeon (KR)

Correspondence Address: BIRCH STEWART KOLASCH & BIRCH, PO BOX 747, FALLS CHURCH, VA, 22040-0747, US

(21) Appl. No.: 10/508,524

(22) PCT Filed: Mar. 21, 2003
(86) PCT No.: PCT/KR03/00558

(30) Foreign Application Priority Data
Mar. 22, 2002 (KR) 10-2002-0015627

(51) Int. Cl. A61K 38/04; A61K 31/4025; C07D 49/02

(52) U.S. Cl. 514/19; 514/422; 548/527

(57) ABSTRACT

The present invention relates to crystalline forms of (2S)-N-5-[amino(imino)methyl]-2-thienyl-methyl-1-(2R)-2-[(carboxymethyl)amino]-3,3-diphenylpropanoyl-2-pyrrolidinecarboxamide nH₂O.

### TECHNICAL FIELD

The present invention relates to crystalline forms of (2S)-N-5-[amino(imino)methyl]-2-thienylmethyl-1-(2R)-2-[(carboxymethyl)amino]-3,3-diphenylpropanoyl-2-pyrrolidinecarboxamide.nH₂O represented by the following Formula (1): 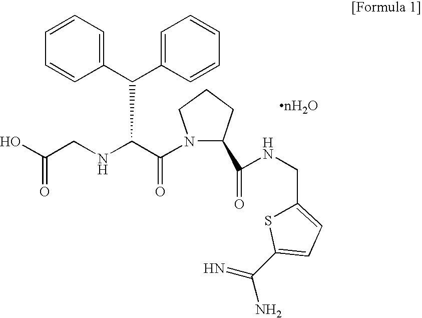
wherein n is the number of combined water per molecule and represents 0, 1, 3, 4, 6 or 7.5.

### BACKGROUND OF THE INVENTION

The free compound of Formula (1), i.e., compound to which acids were not added, and pharmaceutically acceptable salts, hydrates, solvates, and isomers thereof are the subjects of Korean Patent Laid-Open Publication No. 2000-047461 and WO 0039124, and may be effectively used as new thrombin inhibitors.

The physical property of a drug has a huge effect on production and development process of its raw drug and development process of its final product. A drug may be roughly divided into crystalline form and amorphous form according to its crystallinity. Some drugs may be obtained in both crystalline form and amorphous form, while other drugs may be obtained only in either crystalline form or amorphous form. Crystalline form and amorphous form may exhibit large difference in physicochemical properties. For instance, there is a report that an oral absorption rate or bioavailability is different in some drugs because solubility and dissolution rate are different depending on whether the drugs are in crystalline form or amorphous form (see, Pharmaceutical Solids: A Strategic Approach to Regulatory Considerations, Stephen Byrn et al, Pharmaceutical Research, 945, 12(7), 1995). Bioavailability of a drug is directly related to its effect and side effect. In other to say, to obtain the desired effect of a drug, a certain desired blood concentration should be reached. If the blood concentration becomes unduly high, a side effect or toxicity is accompanied. Bioavailability may be improved by selecting a suitable crystalline form. Thus, the crystalline form of a drug should be identified in the course of development and approval of the drug.

Except special cases, it is easy to obtain a drug having crystallinity in the process of its research and development. A report shows that the crystallinity of a drug may be an important advantage because in the final step for producing the drug, the drug may be purely obtained through recrystallization that is a relatively easy purification process, and a drug having crystallinity, whose physicochemical properties may be easily identified, is advantageous even in the quality control of its product process (see, An integrated approach to the selection of optimal salt form for a new drug candidate, Abu T. M. Serajuddin et al, International Journal of Pharmaceutics, 209, 105, 1994). On the other hand, some drugs having crystallinity may have polymorphism. An article reported that generally speaking, in case that the crystalline structure of a drug is different, its solubility or other physical properties may be different, and the crystalline form of a drug may be changed under certain conditions [Pharmaceutical Solids: A Strategic Approach to Regulatory Considerations, Stephen Byrn et al., Pharmaceutical Research, 945, 12(7), 1995]. Therefore, in case that a drug has polymorphism, to obtain purely all crystalline forms of the drug and to discover physical properties of each form are very important in the development and production of the drug.

### BRIEF SUMMARY OF THE INVENTION

Accordingly, the present inventors have found crystalline forms useful as thrombin inhibitors by obtaining various crystalline forms from the free compound of the above Formula (1) and identifying their physical properties.

Therefore, the purpose of the present invention is to provide crystalline forms of (2S)-N-5-[amino(imino)methyl]-2-thienylmethyl-1-(2R)-2-[(carboxymethyl)amino]-3,3-diphenylpropanoyl-2-pyrrolidinecarboxamide.nH₂O represented by the following Formula (1): 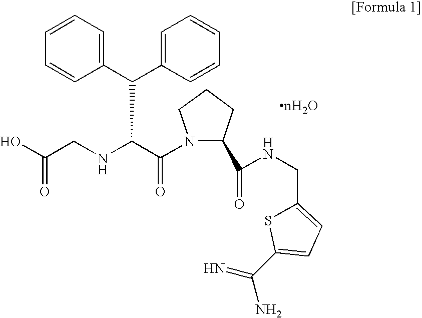
wherein n is the number of combined water per molecule and represents 0, 1, 3, 4, 6 or 7.5.

### BRIEF DESCRIPTION OF DRAWINGS

FIG. 1 is a powder X-ray diffraction diagram of the crystalline Form I of (2S)-N-5-[amino(imino)methyl]-2-thienylmethyl-1-(2R)-2-[(carboxymethyl)amino]-3,3-diphenylpropanoyl-2-pyrrolidinecarboxamide.

FIG. 2 is a powder X-ray diffraction diagram of the crystalline Form II of (2S)-N-5-[amino(imino)methyl]-2-thienylmethyl-1-(2R)-2-[(carboxymethyl)amino]-3,3-diphenylpropanoyl-2-pyrrolidinecarboxamide.

FIG. 3 is a powder X-ray diffraction diagram of the crystalline Form III of (2S)-N-5-[amino(imino)methyl]-2-thienylmethyl-1-(2R)-2-[(carboxymethyl)amino]-3,3-diphenylpropanoyl-2-pyrrolidinecarboxamide.

FIG. 4 is a powder X-ray diffraction diagram of the crystalline Form IV of (2S)-N-5-[amino(imino)methyl]-2-thienylmethyl-1-(2R)-2-[(carboxymethyl)amino]-3,3-diphenylpropanoyl-2-pyrrolidinecarboxamide.

FIG. 5 is a powder X-ray diffraction diagram of the crystalline Form V of (2S)-N-5-[amino(imino)methyl]-2-thienylmethyl-1-(2R)-2-[(carboxymethyl)amino]-3,3-diphenylpropanoyl-2-pyrrolidinecarboxamide.

FIG. 6 is a powder X-ray diffraction diagram of the crystalline Form VI of (2S)-N-5-[amino(imino)methyl]-2-thienylmethyl-1-(2R)-2-[(carboxymethyl)amino]-3,3-diphenylpropanoyl-2-pyrrolidinecarboxamide.

### DETAILED DESCRIPTION

The free compound of the above Formula (1) may be prepared according to a known method (see, Korean Patent Laid-Open Publication No. 2000-047461 and WO0039124).

The crystalline forms of Formula (1) of the present invention obtained from the above free compound or other crystalline forms exist in the form of anhydride or hydrates having various combined water. Preferably, according to the recrystallization method and the number of combined water, the crystalline Form I (n=7.5), the crystalline Form II (n=4), the crystalline Form III (n=6), the crystalline Form IV (n=3), the crystalline Form V (n=0), and the crystalline Form VI (n=1) may be obtained. For instance, the crystalline Form IV may be obtained by dissolving the free compound of Formula (1) in the mixed solvent of water, and methanol or ethanol while heating and recrystallizing it.

The crystalline Form V may be obtained by drying the crystalline Form IV under vacuum. The crystalline Form VI may be obtained by moisture absorption of the Form V. However, the Form I may be obtained by stirring the Form VI in water. The crystalline Form II may be obtained by drying the Form I under vacuum. And, the Form III may be obtained by moisture absorption of the Form II. Since the molecular weight of the above free compound is 533.65, the theoretical water contents of these hydrates of Formula (1) are 0, 3.3, 9.2, 11.9, 16.8, and 20.2%, to the hydrates of Formula (1) wherein n is 0, 1, 3, 4, 6, and 7.5, respectively. However, it is usual that the water contents of actually obtained samples deviate from the above theoretical values depending on drying condition and drying time in preparation, amount of the surface moisture absorbed at the surface, etc. Therefore, the water content of the hydrate of Formula (1) wherein n is 0, i.e., anhydride of Formula (1), may be 0˜3%, that of the hydrate wherein n is 1 may be 2˜9%, that of the hydrate wherein n is 3 may be 4˜11%, that of the hydrate wherein n is 4 may be 9˜15%, that of the hydrate wherein n is 6 may be 12˜20%, and that of the hydrate wherein n is 7.5 may be 16˜26%. Thus, to identify the crystalline form of Formula (1), the water content should be identified, with conducting the powder X-ray diffraction test.

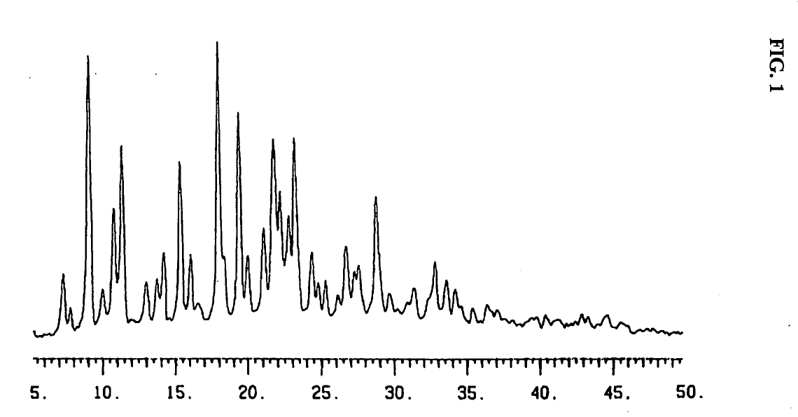

Each crystalline form may be distinguished by characteristic peaks shown at the powder X-ray diffraction test. For example, as shown in Tables 1, 2, 4, 5, 6, and 7, the crystalline Form I has characteristic peaks distinguished from other crystalline forms at 7.3°, 9.1°, 18.0°, and 28.8°, the crystalline Form II at 7.0°, 12.2°, 19.2°, and 20.0°, the crystalline Form III at 10.6°, 19.4°, 20.9°, 21.6°, and 24.4°, the crystalline Form IV at 10.0°, 16.7°, 20.8°, 21.9°, and 26.0°, the crystalline Form V at 15.8°, 18.3°, 20.3°, 20.8°, and 26.5°, the crystalline Form VI at 13.6°, 14.7°, 23.2°, and 27.5°. Further, as shown in FIGS. 1 to 6, it can be confirmed in the power X-ray diffraction diagram that each crystalline form above has a different crystal structure from one another.

A crystalline form may be changed according to storage condition such as relative humidity, etc. Thus, it is important to confirm stability of a crystalline form according to storage condition. Among the above crystalline forms, the crystalline Form VI was identified as a stable hydrate whose crystal structure is not changed under any relative humidity.

Karl-Fischer titrimetry has been widely used for determining the water content in samples (see, Quantitative Chemical Analysis, 4th edition, I. M. Koltmoff et al, 858, The Macmillan Company, 1969). When Karl-Fischer titrimetry was applied to the above crystalline forms, the water content of the crystalline Form VI was proven as 3.5%, which corresponds to the weight ratio of a water molecule when n of Formula (1) is 1. On the other hand, the water content of the crystalline Form 1 was proven as 20.2%, which corresponds to the weight ratio of a water molecule when n of Formula (1) is 7.5.

Moisture included in a sample is not completely removed even if the sample is dried under vacuum. In order to remove moisture completely, various drying agents should be placed with the sample under vacuum. Various kinds of drying agents may be used for the present invention: calcium sulfate, sodium sulfate, calcium chloride, etc. The most widely used drying agent is P₂O₅ (see, MIT Laboratory techniques manual, MIT dept. of Chemistry, 10:43, 1979). If the crystalline Form I is dried under vacuum in a desiccator in which P₂O₅ is used as a drying agent, the moisture included in the crystalline form can be removed. Then, it is confirmed by the power X-ray diffraction test that the crystalline form was changed, and the changed form is identified as the crystalline Form II. The crystalline Form II became stable by adsorbing moisture and its water content is 10.8% that corresponds to the weight ratio of 4 water molecules. If the crystalline Form II is left under highly relative humidity, the form is changed to the crystalline Form III, and its water content is 16.9% that corresponds to the weight ratio of 6 water molecules.

From the above results, it can be seen that the crystalline Form I, Form II and Form III are hydrates wherein n is 7.5, 4, and 6, respectively.

The solvent to be used in recrystallization may be usually available kinds of alcohols, which are alkanes alcohols having the carbon number of 1 to 8, such as methanol, ethanol, propanol, butanol, isopropanol and octanol, etc., but methanol and ethanol are preferable, and methanol is the most preferable, but not limited to them. Furthermore, as a solvent to be used to recrystallize the above free compound, in addition to alcohols exemplified above, water and organic solvents, such as n-hexane, ethylacetate, butylacetate, acetonitril, chloroform, diethylether, acetone, etc., and other usually available solvents may be used. The above free compound may be dissolved or dissolved in heating, by using one solvent or more than one in mixture among the above and may be recrystallized.

If the above several crystalline forms are dissolved in alcohols, a suitable amount of water is added thereto, and the mixture is recrystallized, the crystalline Form IV, another crystalline form, may be obtained. The X-ray crystal structure method identified the crystalline form as hydrate wherein n is 3. The crystalline Form IV was dried under vacuum in the presence of P₂O₅ to obtain the crystalline Form V which is anhydride. The crystalline Form V is changed into the crystalline Form VI by absorbing moisture. The crystalline Form VI has 3.5% of water content, and is stable hydrate wherein n is 1.

The stress stability test showed that the crystalline form of the compound of Formula (1) above is physicochemically more stable than the amorphous form. The amorphous form showed a residual content of only 87% as well as discoloration after 4 weeks' storage, especially at 70° C. However, the crystalline Form I and IV were stable without discoloration.

As Korean patent Laid-Open Publication No. 2000-047461 and WO0039124 are disclosed, the free compound of Formula (1) of the present invention is effectively used as a thrombin inhibitor. And, its crystalline forms are also useful as thrombin inhibitors.

Below, the present invention will be explained in more detail with reference to the following examples, comparative examples, and test examples. However, it should be understood that these examples have been described as preferred specific embodiments of the present invention, and are not intended to limit the scope of the present invention in any way. Other aspects of this invention will be apparent to those skilled in the art to which the present invention pertains.

### EXAMPLES

### Example 1

### Preparation of the crystalline Form II of (2S)-N-5-[amino(imino)methyl]-2-thienylmethyl-1-(2R)-2-[(carboxymethyl)amino]-3,3-diphenylpropanoyl-2-pyrrolidinecarboxamide

The crystalline Form I prepared in the following example 8 was dried under vacuum in the presence of P₂O₅ for one day and then placed at the relative humility of 75% for one day to obtain the titled crystalline Form II.

### Example 2

### Preparation of the crystalline Form III of (2S)-N-5-[amino(imino)methyl]-2-thienylmethyl-1-(2R)-2-[(carboxymethyl)amino]-3,3-diphenylpropanoyl-2-pyrrolidinecarboxamide

The crystalline Form II prepared in Example 1 above was placed at the relative humidity of 93% for one day, and then moved and placed at the relative humidity of 64% for one day to obtain the titled crystalline Form III.

### Example 3

### Preparation of the crystalline Form IV (1) of (2S)-N-5-[amino(imino)methyl]-2-thienylmethyl-1-(2R)-2-[(carboxymethyl)amino]-3,3-diphenylpropanoyl-2-pyrrolidinecarboxamide

The free compound (1 g) of (2S)-N-5-[amino(imino)methyl]-2-thienylmethyl-1-(2R)-2-((carboxymethyl)amino]-3,3-diphenylpropanoyl-2-pyrrolidinecarboxamide was placed into a glass container and then methanol (5.0 ml) was added thereto. While stirring, the mixture was heated to obtain a clear solution. Water (0.5 ml) was added to the solution and then the solution was cooled at room temperature. White crystals were obtained therefrom. The crystals were filtered and then washed with water. They were dried in the air (0.85 g, yield 85%).

### Example 4

### Preparation of the crystalline Form IV (2) of (2S)-N-5-[amino(imino)methyl]-2-thienylmethyl-1-(2R)-2-[(carboxymethyl)amino]-3,3-diphenylpropanoyl-2-pyrrolidinecarboxamide The free compound (1 g) of (2S)-N-5-[amino(imino)methyl]-2-thienylmethyl-1-(2R)-2-[(carboxymethyl)amino]-3,3-diphenylpropanoyl-2-pyrrolidinecarboxamide was placed into a glass container and dissolved by adding methanol (6.0 milliliter), water (1.5 milliliter), and 6N hydrochloric acid solution (0.65 milliliter). Thereafter, 10 N solution of sodium hydroxide (0.2 milliliter) was added thereto and stirred. After 10 N solution of sodium hydroxide (0.4 milliliter) was further added thereto, the solution was placed at room temperature to obtain white needle form crystals. The crystals were filtered, washed with water, and then dried in air (0.8 g, yield 80%).

### Example 5

### Preparation of the crystalline Form V of (2S)-N-5-[amino(imino)methyl]-2-thienylmethyl-1-(2R)-2-[(carboxymethyl)amino]-3,3-diphenylpropanoyl-2-pyrrolidinecarboxamide

The crystalline Form IV prepared in Example 3 or 4 was dried under vacuum in the presence of P₂O₅ for one day to obtain the titled crystalline Form V.

### Example 6

### Preparation of the crystalline Form VI (1) of (2S)-N-5-[amino(imino)methyl]-2-thienylmethyl-1-(2R)-2-[(carboxymethyl)amino]-3,3-diphenylpropanoyl-2-pyrrolidinecarboxamide

The crystalline Form V prepared in Example 5 was placed for one day at the relative humidity of 53% to obtain the titled crystalline Form VI.

### Example 7

### Preparation of the crystalline Form VI (2) of (2S)-N-5-[amino(imino)methyl]-2-thienylmethyl-1-(2R)-2-[(carboxymethyl)amino]-3,3-diphenylpropanoyl-2-pyrrolidinecarboxamide

The crystalline Form V prepared in Example 5 was placed in a glass container, and nitrogen saturated with water was passed through the container for one hour to obtain the titled crystalline Form VI.

### Example 8

### Preparation of the crystalline Form 1 of (2S)-N-5-[amino(imino)methyl]-2-thienylmethyl-1-(2R)-2-[(carboxymethyl)amino]-3,3-diphenylpropanoyl-2-pyrrolidinecarboxamide

Water was added to all the crystalline forms except the crystalline Form I and the mixture was stirred for one hour or more to obtain the titled crystalline Form I.

### Comparative Example 1

### Preparation of the amorphous form of (2S)-N-5-[amino(imino)methyl]-2-thienylmethyl-1-(2R)-2-[(carboxymethyl)amino]-3,3-diphenylpropanoyl-2-pyrrolidinecarboxamide

The crystalline Form III obtained at Example 2 was dried under vacuum in the presence of P₂O₅ for two days to obtain the titled amorphous form.

### Test Example 1

### Powder X-ray diffraction test of the free compound of (2S)-N-5-[amino(imino)methyl]-2-thienylmethyl-1-(2R)-2-[(carboxymethyl)amino]-3,3-diphenylpropanoyl-2-pyrrolidinecarboxamide

Each 40 mg of the crystalline Form 1 and the crystalline Form IV prepared in Example 8 and Example 3 or 4 was thinly coated onto a sample holder, and thereafter the powder x-ray diffraction test was conducted thereto according to the following conditions. By using Rigaku Geigeflex D/max-III C apparatus, the test was conducted at 35 kV, 20 mA.
Scan speed(2E) 5°/minute
Sampling time 0.03 sec
Scan mode: continuous
Cu-target (Ni filter)

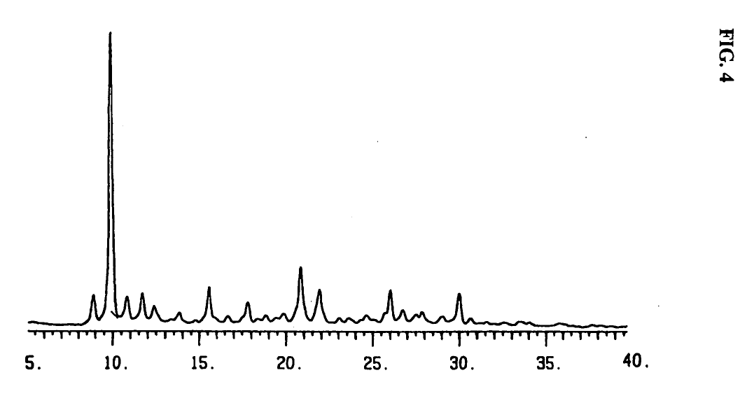

The results of the powder X-ray diffraction test to the crystalline Form I and Form IV are shown in FIGS. 1 and 4. The positions of peaks shown in the above figures are listed at Tables 1 and 2. As shown in each result, each crystalline form has different crystallinity.

TABLE 1
Peaks of the powder X-ray diffraction of the crystalline Form I of
(2S)-N-5-[amino(imino)methyl]-2-thienylmethyl-1-(2R)-2-
[(carboxymethyl)amino]-3,3-diphenylpropanoyl-2-pyrrolidinecarboxamide
peak
2θ

7.319
7.81
9.117
10.02
10.808
11.397
13.01
13.732
14.192
15.346
16.05
16.539
18.003
19.425
20.01
21.111
21.832
22.226
22.802
23.212
24.368
24.781
25.289
26.129
26.698
27.257
27.568
28.802
29.632
30.867

TABLE 2
Peaks of the powder X-ray diffraction of the crystalline Form IV of
(2S)-N-5-[amino(imino)methyl]-2-thienylmethyl-1-(2R)-2-
[(carboxymethyl)amino]-3,3-diphenylpropanoyl-2-pyrrolidinecarboxamide
peak
2θ

8.923
9.966
10.845
11.727
12.395
13.335
13.843
14.778
15.591
16.686
17.819
18.364
18.85
19.419
19.871
20.835
21.92
23.06
23.617
24.629
25.09
26.017
26.746
27.522
27.872
29.043
30
30.649
31.547

### Test Example 2

### Powder X-ray diffraction test during moisture absorption and dehumidification of the crystalline Form I of (2S)-N-5-[amino(imino)methyl]-2-thienylmethyl-1-(2R)-2-[(carboxymethyl)amino]-3,3-diphenylpropanoyl-2-pyrrolidinecarboxamide

40 mg of the above crystalline Form I was thinly coated onto a sample holder. And, immediately after the sample was dried under vacuum in the presence of P₂O₅, and after the sample was placed for moisture absorption at each relative humidity of 33%, 53%, 64%, 75%, and 93% for two days or more, respectively, the powder X-ray diffraction test was conducted on the sample according to the conditions represented in above Test example 1 to observe change of the crystalline formi during moisture absorption. While lowering the relative humidity, the same test was repeated to observe change of the crystalline form during dehumidification.

In order to obtain each relative humidity above, as shown in the table below, saturated aqueous solutions of salts were prepared, then placed in a desiccator, and the desiccator was sealed.

TABLE 3

| | |
|---|---|
| Relative Humidity 33% | MgCl₂ saturated aqueous solution |
| Relative Humidity 53% | Mg(NO₃)₂.6H₂O saturated aqueous |
| | solution |
| Relative Humidity 64% | NaNO₂ saturated aqueous solution |
| Relative Humidity 75% | NaCl saturated aqueous solution |
| Relative Humidity 93% | KNO₃ saturated aqueous solution |

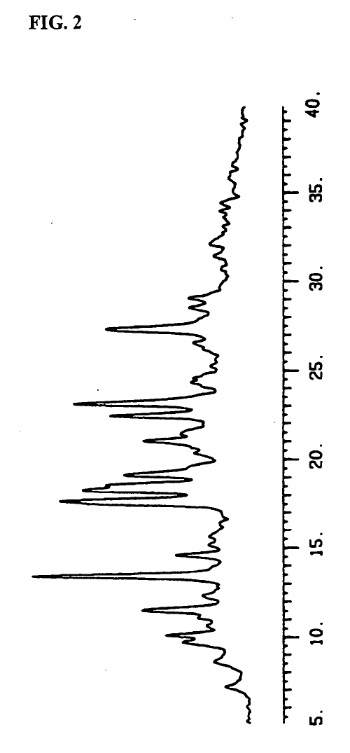

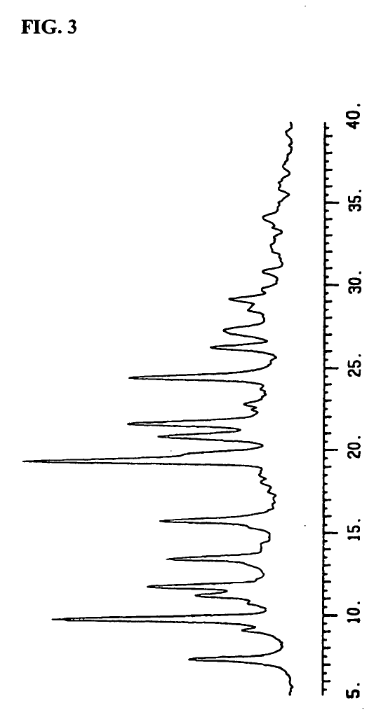

The results of the powder X-ray diffraction test of the crystalline Form II exhibited immediately after the vacuum drying to the relative humidity of 75%, and of the crystalline Form III exhibited at the relative humidity of 64%˜33% during dehumidification are provided in FIGS. 2 and 3, respectively. The positions of peaks shown at the figures are listed at the following Tables 4 and 5. Each result shows that each crystalline form has different crystallinity.

TABLE 4
Peaks of the powder X-ray diffraction of the crystalline Form II of
(2S)-N-5-[amino(imino)methyl]-2-thienylmethyl-1-(2R)-2-
[(carboxymethyl)amino]-3,3-diphenylpropanoyl-2-pyrrolidinecarboxamide
peak
2θ

7.012
7.822
9.739
10.607
11.43
12.15
13.841
15.17
16.384
17.122
17.802
19.198
20.052
20.954
21.882
22.68
23.713
24.837
25.438
25.902
26.387
28.046
28.501
28.935
29.304
29.856
30.866
31.405
32.098
33.016

TABLE 5
Peaks of the powder X-ray diffraction of the crystalline Form III of
(2S)-N-5-[amino(imino)methyl]-2-thienylmethyl-1-(2R)-2-
[(carboxymethyl)amino]-3,3-diphenylpropanoyl-2-pyrrolidinecarboxamide
peak
2θ

7.335
9.09
9.808
10.601
11.203
11.761
13.44
15.245
15.755
19.389
20.86
21.629
24.436
26.236
27.159
29.123
29.73
30.763

### Test Example 3

### Powder X-ray diffraction test during moisture absorption and dehumidification of the crystalline Form IV of (2S)-N-5-[amino(imino)methyl]-2-thienylmethyl-1-(2R)-2-[(carboxymethyl)amino]-3,3-diphenylpropanoyl-2-pyrrolidinecarboxamide

40 mg of the above crystalline Form IV was thinly coated onto a sample holder. Immediately after the sample was dried under vacuum in the presence of P₂O₅, and after the sample was placed for moisture absorption at each relative humidity of 33%, 53%, 64%, 75%, and 93% for two days or more, respectively, the powder X-ray diffraction test of the sample was conducted according to the conditions represented in Test example 1 above to observe change of the crystalline form during moisture absorption. While lowering the relative humidity, the same test was repeated to observe change of the crystalline form during dehumidification.

In order to obtain each relative humidity above, as shown in Table 3 of Test example 2, saturated aqueous solutions of salts were prepared and then placed in a desiccator, and the desiccator was sealed.

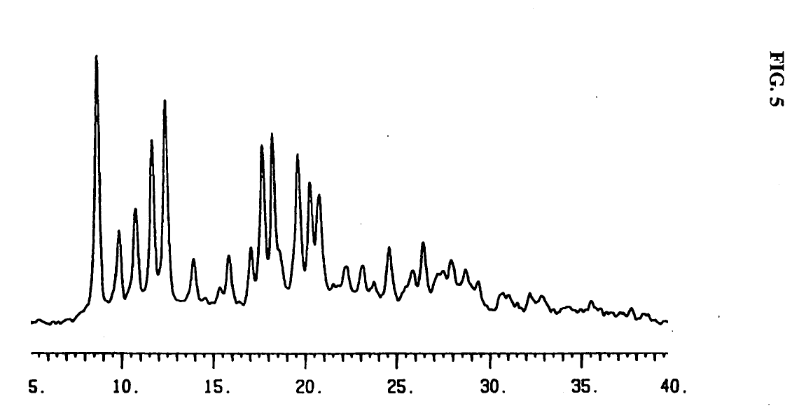

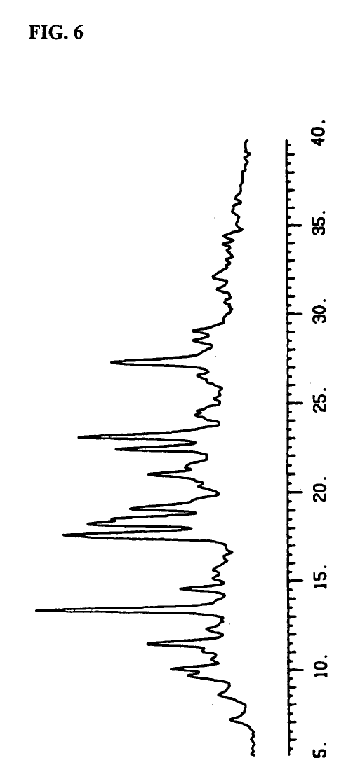

The results of the powder X-ray diffraction test of the crystalline Form V exhibited immediately after the vacuum drying and of the crystalline Form VI exhibited after moisture absorption get started are provided in FIGS. 5 and 6, respectively. The positions of peaks shown at the figures are listed in the following Tables 6 and 7. Each result shows that each crystalline form has different crystallinity.

TABLE 6
Peaks of the powder X-ray diffraction of the crystalline Form V of
(2S)-N-5-[amino(imino)methyl]-2-thienylmethyl-1-(2R)-2-
[(carboxymethyl)amino]-3,3-diphenylpropanoyl-2-pyrrolidinecarboxamide
peak
2θ

8.739
9.878
10.789
11.716
12.451
13.965
14.567
15.368
15.858
17.093
17.757
18.296
19.674
20.319
20.799
22.227
23.112
23.742
24.596
25.873
26.458
27.502
27.935
28.68
29.358

TABLE 7
Peaks of the powder X-ray diffraction of the crystalline Form VI of
(2S)-N-5-[amino(imino)methyl]-2-thienylmethyl-1-(2R)-2-
[(carboxymethyl)amino]-3,3-diphenylpropanoyl-2-pyrrolidinecarboxamide
peak
2θ

8.042
8.718
10.231
10.78
11.668
12.445
13.56
14.682
15.222
15.864
16.5
17.084
17.814
18.698
19.225
19.659
20.327
21.14
22.541
23.246
24.656
25.275
25.86
26.636
27.453
28.584
29.147
29.755
30.793

### Test example 4

### Stress stability test for the amorphous form, and the crystalline Form I and Form VI

In order to compare physicochemical stability among the crystalline Form VI, the crystalline Form I, and the amorphous form prepared in Examples 7, 8, and Comparative Example 1, the stress stability test was conducted by placing their samples at the temperatures of −20° C., 50° C., and 70° C. for 4 weeks. The results are summarized at the following Table 8.

TABLE 8

| | | Form I | Form VI | Amorphous form |
|---|---|---|---|---|
| | | Color | | |
| | | Ivory | Ivory | Yellow |
| Residual rate | −20° C. | 99% | 101% | 96% |
| after 4 weeks | 50° C. | 99% | 99% | 96% |
| Residual rate | 70° C. | 100% | 100% | 87% |

### Industrial Applicability

As shown from the above results, the crystalline Form I and Form VI exhibited remarkably superior stability over the amorphous form. The amorphous form did not show any change in appearance at −20° C. and 50° C., but showed a residual rate of 96% after 4 weeks. At 70° C., the amorphous form showed a residual rate of 87% as well as a change in appearance. Therefore, it can be seen that the crystalline forms according to the present invention show superior physicochemical stability over the amorphous form.

1. Crystalline forms of (2S)-N-5-[amino(imino)methyl]-2-thienylmethyl-1-(2R)-2-[(carboxymethyl)amino]-3,3-diphenylpropanoyl-2-pyrrolidinecarboxamide.nH₂O represented by the following Formula (1): 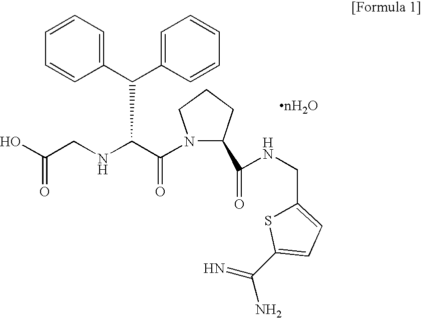
wherein n is the number of combined water per molecule and represents 0, 1, 3, 4, 6, or 7.5:

2. The crystalline forms of claim 1 wherein n represents 1.

3. The crystalline forms of claim 1 or claim 2 wherein X-ray diffraction angles are 13.6°, 14.7°, 23.2°, and 27.5°.

4. The crystalline forms of claim 1 wherein water content is 2 to 9%.

5. The crystalline forms of claim 1 wherein n represents 4.

6. The crystalline forms of claim 1 or claim 5 wherein X-ray diffraction angles are 7.0°, 12.2°, and 19.2°.

7. The crystalline forms of claim 1 wherein water content is 9 to 15%.

8. The crystalline forms of claim 1 wherein n represents 7.5.

9. The crystalline forms of claim 1 or claim 8 wherein X-ray diffraction angles are 7.3°, 9.1°, 18.0°, and 28.8°.

10. The crystalline forms of claim 1 wherein water content is 16 to 26%.
